(19) United States
(12) Patent Application Publication
(10) Pub. No.: US 2026/0232186 A1
(43) Pub. Date: Aug. 13, 2026

(54) CONTACT LENS VIEWING DEVICE AND METHOD

(71) Applicant: Edwin Ryan, St. Paul, MN (US)

(72) Inventor: Edwin Ryan, St. Paul, MN (US)

(21) Appl. No.: 19/532,727

(22) Filed: Feb. 6, 2026

Related U.S. Application Data
(60) Provisional application No. 63/755,909, filed on Feb. 7, 2025.

(51) Int. Cl. A61B 3/125

(52) U.S. Cl. CPC A61B 3/125

(57) ABSTRACT

An ophthalmologic visualization system and associated methods are shown. Examples include components and methods for use with a contact lens system. Example configurations and methods as described include a contact lens holder with a side surface that engages a plurality of cannulas placed in a patient's eye. A combination of the side surface of the contact lens holder and the plurality of cannulas holds the contact lens holder in place.

### CLAIM OF PRIORITY

This patent application claims the benefit of priority, under 35 U.S.C. § 119(e), to U.S. Provisional Patent Application Ser. No. 63/755,909, entitled “CONTACT LENS VIEWING DEVICE AND METHOD,” filed on Feb. 7, 2025, which is hereby incorporated by reference herein in its entirety.

### TECHNICAL FIELD

This invention relates to intraoperative visualization instruments typically used for surgical procedures such as surgery of the eye.

### BACKGROUND

Ophthalmological surgery often requires visualization of an interior of a patient's eye. In selected procedures, a microscope is utilized to better view small structures and detail within an eye. Prior to roughly 1995, contact lenses were used that afforded a very restricted view or the retina during vitrectomy. Then the AVI system, using a wide-angle contact lens, was introduced and vastly improved viewing during vitrectomy. This system required a skilled assistant to hold the lens properly to allow a clear view, a skill that was difficult to acquire, A non-contact viewing lens was developed that can be used adjacent to a patient's eye in conjunction with the microscope for many viewing needs, and this required no skilled assistant. A non-contact lens, as the name implies, does not touch the patient's eye during use. Drawbacks of non-contact lens systems includes limited angle of visualization within the eye, and glare from additional optical interfaces. The wide angle contact lens gives a superior, clearer view, and a system that would hold in place such a lens so that it would not require a highly skilled assistant would be desirable.

### BRIEF DESCRIPTION OF THE DRAWINGS

FIG. 1 shows selected components of an ophthalmologic visualization system according to an embodiment of the invention.

FIG. 2 shows selected components of an ophthalmologic visualization system according to an embodiment of the invention.

FIG. 3 shows selected components of an ophthalmologic visualization system according to an embodiment of the invention.

FIG. 4 shows selected components of an ophthalmologic visualization system according to an embodiment of the invention.

FIG. 5 shows a diagram of an ophthalmologic visualization system in use according to an embodiment of the invention.

FIG. 6 shows a contact lens holder according to an embodiment of the invention.

FIG. 7A shows a top view and a cross section side view of a contact lens holder according to an embodiment of the invention.

FIG. 7B shows a cross section side view of the contact lens holder from FIG. 7A according to an embodiment of the invention.

FIG. 7C shows another cross section side view of the contact lens holder from FIG. 7A according to an embodiment of the invention.

FIG. 8 shows a contact lens and a contact lens holder according to an embodiment of the invention.

FIG. 9A shows an eye with a number of cannulas after placement in the eye according to an embodiment of the invention.

FIG. 9B shows the eye from FIG. 9A along with an indicated placement of a contact lens holder according to an embodiment of the invention.

FIG. 10A shows an ophthalmologic visualization system according to an embodiment of the invention.

FIG. 10B shows a cross section of the ophthalmologic visualization system from FIG. 10A according to an embodiment of the invention.

FIG. 10C shows another ophthalmologic visualization system according to an embodiment of the invention.

FIG. 11 shows a diagram of another ophthalmologic visualization system in use according to an embodiment of the invention.

FIG. 12 shows an example method of visualizing an interior of a patient's eye according to an embodiment of the invention.

### DETAILED DESCRIPTION

In the following detailed description, reference is made to the accompanying drawings which form a part hereof, and in which is shown, by way of illustration, specific embodiments in which the invention may be practiced. In the drawings, like numerals describe substantially similar components throughout the several views. These embodiments are described in sufficient detail to enable those skilled in the art to practice the invention. Other embodiments may be utilized and structural, or logical changes, etc. may be made without departing from the scope of the present invention.

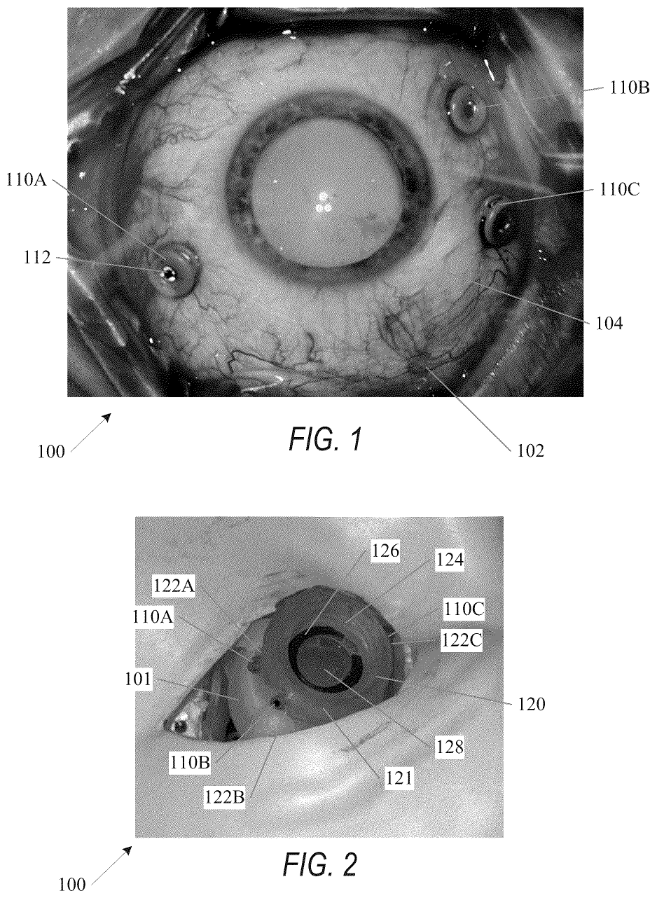

FIG. 1 shows an ophthalmologic visualization system 100. A patient's eye 102 is shown, with a plurality of cannulas 110A-110C placed in a sclera 104 of the patient's eye 102. In the example of FIG. 1, three cannulas 110A, 110B, and 110C are placed. Although three cannulas are used in this example, the invention is not so limited. More than three cannulas may also be used. At least three cannulas 110A-110C provides an advantage of location stability as discussed in more detail below. However, fewer than three cannulas also provide a level of location stability, and fewer than three cannula examples are also within the scope of the invention. Each cannula 110 includes a passage 112 that may be used for a number of different surgical functions including, but not limited to, infusion, introduction of surgical instrumentation, lighting, etc.

FIG. 2 shows a picture of a model of a patient's eye 101 to illustrate different features of the ophthalmologic visualization system 100. The three cannulas 110A-110C are shown again in the context of the model eye 101. A contact lens holder 120 is shown engaging the three cannulas 110A-110C. The contact lens holder 120 includes a holder body 121 with a top opening 124 in the holder body 121 sized to fit closely around a bottom portion of a viewing lens (not shown in FIG. 2). The top opening 124 shown in FIG. 2 includes a shelf 126 upon which the bottom portion of the viewing lens will rest, as discussed in more detail below. A central window 128 allows viewing into the eye 101 when the contact lens is in place.

The holder body 121 further includes a plurality of notches 122A-122C in a side of the holder body 121, adjacent to a bottom surface of the contact lens holder 120. The plurality of notches are shaped to substantially match a lateral shape of the plurality of cannulas 110A-110C. As shown in FIG. 2, the plurality of notches 122A-122C are also arranged to fit closely with all of the plurality of cannulas 110A-110C at the same time. This arrangement centers and holds the contact lens holder 120 in place with the central window 128 centered over a cornea of the eye 101.

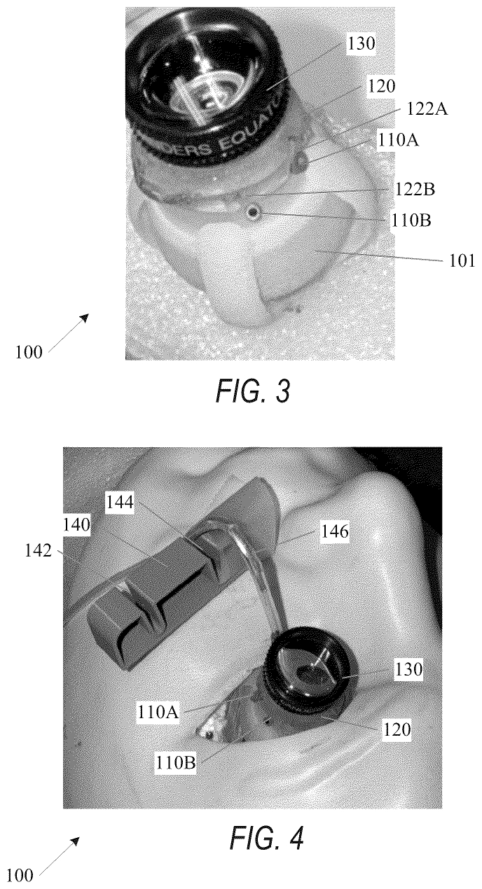

FIG. 3 shows the ophthalmologic visualization system 100 including a viewing lens 130. In the example shown, the viewing lens 130 is a contact lens. As noted above, a contact viewing lens 130 provides improved viewing over a non-contact viewing lens. The viewing lens 130 is shown located within the top opening 124 in the holder body 121. The plurality of notches 122A-122C are again shown engaging the plurality of cannulas 110A-110C to hold the contact lens holder 120 in place. The contact lens holder 120 further holds the viewing lens 130 in place. Because of the centering and orienting properties of the plurality of notches 122A-122C and the plurality of cannulas 110A-110C, the contact viewing lens 130 can be used with minimal assistant aid, or without the need for a skilled assistant to manually orient the contact viewing lens 130.

FIG. 4 shows the ophthalmologic visualization system 100 with an additional tubing holder 140. The tubing holder 140 includes at least one slot 142 to hold tubing for additional functionality. Examples of tubing include, but are not limited to, infusion tubing, fiber optic lighting, etc. In the example of FIG. 4, the tubing holder 140 further includes a second slot 144, allowing the tubing holder 140 to manage two different types of tubing at the same time. A first tube 146 is shown being held by the second slot 144. In one example, the first tube 146 is coupled to one of the plurality of cannulas 110A-110C. In one example, the first tube 146 is coupled to a post formed in the holder body 121 to further aid in stabilizing the contact lens holder 120. When utilizing a post formed in the holder body 121, the first tube may be merely structural, and not used to deliver any infusion, lighting, etc. By using one of two slots 142, 144 to hold the lens holder 120 by a post, the other of the two slots 142, 144 will still be available to hold a functional infusion tube, fiber optic line, etc.

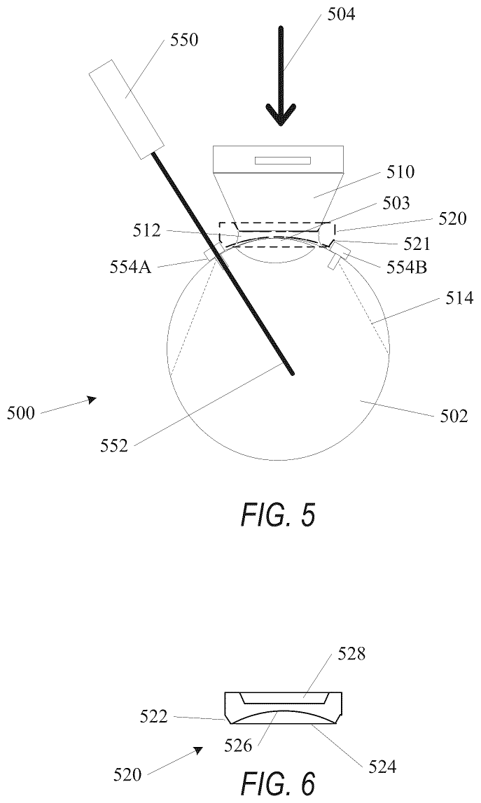

FIG. 5 shows one example of a procedure 500 using ophthalmologic visualization systems as described. An eye 502 is shown. A viewing direction 504 from a microscope (not shown) is illustrated in FIG. 5. A viewing lens 510 similar to viewing lenses described in other examples above is shown. The viewing lens 510 contacts the eye 502. In one example, a viscoelastic fluid 512 is used at an interface 503 between the viewing lens 510 and the eye 502 to provide a continuous transparent medium at the interface 503. In contrast to non-contact lens systems, a contact viewing lens 510 as shown in FIG. 5, provides a wider angle of view 514, and is desirable in many procedures. An instrument 550 such as a probe, suction, light source, etc. is shown with a functional end 552 entering the eye 502 through a cannula 554A. The viewing lens 510 is utilized to visualize a procedure using the functional end 552 of the instrument 550.

A contact lens holder 520 is shown located between the viewing lens 510 and the eye 502. The contact lens holder 520 is shown in FIG. 5 engaging a first cannula 554A and a second cannula 554B. Although two cannulas 554A, 554B are shown, three or more cannulas are also within the scope of the invention. Three cannulas provides a stability advantage, as three point will define one and only one circle around a cornea. The use of three cannulas therefore provides a stable fixed location for the contact lens holder 520. In the example of FIG. 5, a portion of sides of the contact lens holder 520 are shown with a bevel 521 that substantially matches an angled side surface of the cannulas 554A, 554B. The bevel 521 aids in stability of the contact lens holder 520 and helps prevent a possibility of sides of the contact lens holder 520 undermining one or more of the cannulas 554A, 554B and causing them to begin to pull out of the eye 502.

FIG. 6 shows the contact lens holder 520 from FIG. 5 in more detail. The contact lens holder 520 includes a top opening 528 sized to fit closely around a bottom portion of the viewing lens 510. The contact lens holder 520 further includes a bottom surface 524 to contact the patient's eye 502. A cavity 526 is shown in the contact lens holder 520 to provide space for the cornea of the patient's eye 502. In one example, the cavity 526 is close fitting to the cornea of the patient's eye 502. In other examples, the cavity 526 includes excess space for an interfacial fluid, such as the viscoelastic fluid 512 shown in FIG. 5. As noted above, side surface 522 includes a bevel 521 that substantially matches an angled side surface of the cannulas 554A, 554B. In one example the side surface 522 includes a plurality of notches as discussed in examples above. In one example, only the plurality of notches include the bevel 521 that substantially matches an angled side surface of the cannulas 554A, 554B.

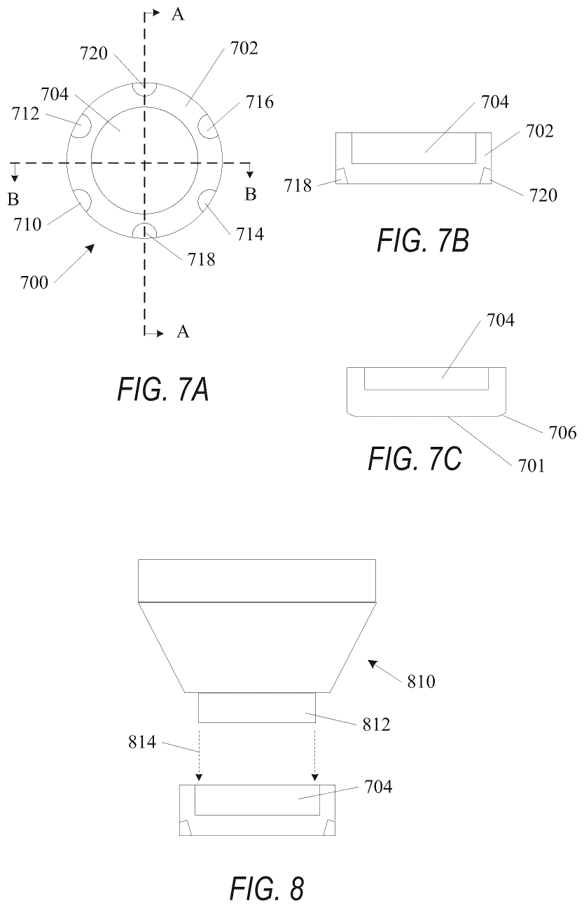

FIG. 7A shows a top view of another example contact lens holder 700. The contact lens holder 700 includes a holder body 702 with a top opening 704 in the holder body 702 sized to fit closely around a bottom portion of a viewing lens. The holder body 702 further includes a plurality of notches in a side of the holder body 702, adjacent to a bottom surface of the holder body 702. In the example of FIG. 7 the plurality of notches includes a first notch 710, a second notch 712, a third notch 714, a fourth notch 716, a fifth notch 718 and a sixth notch 720.

In use, often two cannulas are placed on a temporal side of a patient's eye and a single cannula is placed on a nasal side of the patient's eye. By including an arrangement with the first notch 710, the second notch 712, the third notch 714, and the fourth notch 716, the contact lens holder 700 can be utilized on either a left or right eye of a patient when three cannulas are placed as described above. In one example, on a left eye, the first notch 710 and the second notch 712 are used to engage placed cannulas on a temporal side of a patient's eye and the third notch 714 is used to engage a placed cannula on a nasal side of the patient's eye. Similarly, on a right eye, the third notch 714 and the fourth notch 716, are used to engage placed cannulas on a temporal side of a patient's eye and the first notch 710 is used to engage a placed cannula on a nasal side of the patient's eye. In one example, the fifth notch 718 and the sixth notch 720 are included to provide cut out space for additional instrumentation to access the eye.

FIG. 7B shows a cross section of the contact lens holder 700 along line A-A from FIG. 7A. The fifth notch 718 and the sixth notch 720 are shown including a bevel to engage a lateral side of cannulas as discussed in examples above. FIG. 7C shows a cross section of the contact lens holder 700 along line B-B from FIG. 7A. A bottom surface 701 of the contact lens holder 700 includes a curved edge 706 to minimize sharp edges contacting a patient's eye.

FIG. 8 shows a contact viewing lens 810 aligned with the top opening 704 of the holder body 702. As indicated by arrows 814, the contact viewing lens 810 is adapted to engage the top opening 704 and be held in place within the top opening 704 of the holder body 702. A bottom portion 812 of the contact viewing lens 810 is dimensioned to fit within closely within the top opening 704 of the holder body 702. As discussed above, selected combinations of the notches are used to engage cannulas placed within the sclera of a patient's eye. In this way, contact lens holders as described provide a simple and effective device to hold a contact viewing lens 810 in position over a patient's eye with minimal assistance from a technician or in some cases without any assistance from a technician.

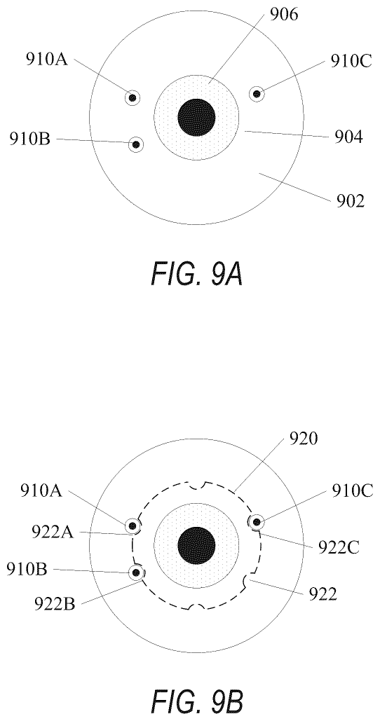

FIG. 9A shows a top view of a patient's eye 902 including a sclera portion 904 and a cornea 906. A plurality of cannulas 910A, 910B and 910C are shown placed about the cornea 906 within the sclera portion 904. In FIG. 9B, an outline 920 of a base of a contact viewing lens is shown. The outline 920 illustrates how a plurality of notches 922 are included similar to examples described above. In the example of FIG. 9B, a first notch 922A engages a first cannula 910A. A second notch 922B engages a second cannula 910B and a third notch 922C engages a third cannula 910C. As shown in FIG. 9B, some notches 922 are not engaged during some procedures. As discussed above, in some examples different notches 922 are utilized depending on whether a left eye or a right eye is under the procedure. In some examples selected notches 922 are only utilized if optional device access is desired.

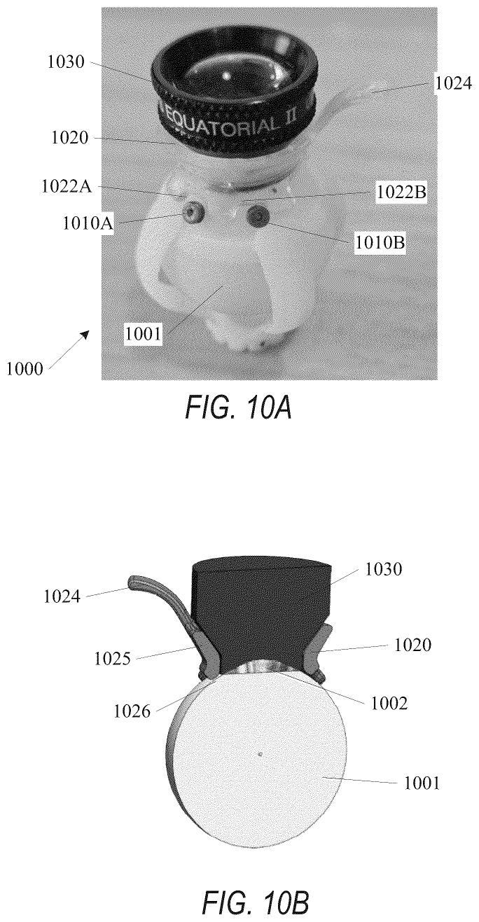

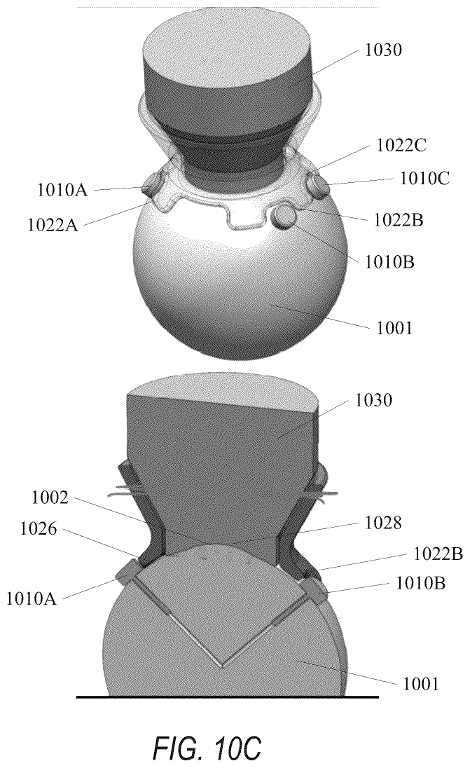

FIG. 10A shows an ophthalmologic visualization system 1000 including many similar features to other ophthalmologic visualization system described above. The ophthalmologic visualization system 1000 is shown in contact with a model of a patient's eye 1001. In one example, the ophthalmologic visualization system 1000 includes a viewing lens 1030. In the example shown, the viewing lens 1030 is a contact lens. As noted above, a contact viewing lens 1030 provides improved viewing over a non-contact viewing lens. The viewing lens 1030 is shown located within the top opening 1025 in a holder body. A plurality of notches 1022A-1022C are shown engaging a plurality of cannulas 1010A-1010C to hold the contact lens holder 1020 in place. The contact lens holder 1020 further holds the viewing lens 1030 in place. Because of the centering and orienting properties of the plurality of notches 1022A-1022C and the plurality of cannulas 1010A-1010C, the contact viewing lens 1030 can be used with minimal assistant aid, or without the need for a skilled assistant to manually orient the contact viewing lens 1030.

A handle 1024 or post is shown in FIG. 10A protruding from a side of the contact lens holder 1020. The handle 1024 can be used for additional stability as discussed in FIG. 4. Also, the handle 1024 can be used to facilitate placement or removal of the contact lens holder 1020 and the contact viewing lens 1030.

FIG. 10B shows a cross section of the ophthalmologic visualization system 1000 from FIG. 10A. The cross section view shows detail of how the top opening 1025 allows the contact viewing lens 1030 to extend towards the patient's eye 1001 such that it makes contact with a cornea 1002 of the patient's eye 1001. A bottom surface of the contact lens holder 1020 is shown in contact with a limbus region 1026 of the patient's eye 1001, adjacent to the cornea 1002. In the example shown, the m surface of the contact lens holder 1020 is rounded where it makes contact with the limbus region 1026 to eliminate any sharp edges that may irritate or damage the limbus region 1026.

FIG. 10C shows another pair of views of the ophthalmologic visualization system 1000. In FIG. 10C, the plurality of notches 1022A-1022C are more clearly shown in one possible geometry that engages the plurality of cannulas 1010A-1010C. FIG. 10C further illustrates the contact viewing lens 1030 extending through the contact lens holder 1020 towards the patient's eye 1001 such that it makes contact with the cornea 1002 of the patient's eye 1001 at interface 1028. Edges of the notches 1022A-1022C are shown making contact with the plurality of cannulas 1010A-1010C to provide centering and support to the contact lens holder 1020, and the contact viewing lens 1030. Two of the plurality of cannulas 1010A and 1010B are shown placed in a sclera region of the patient's eye 1001 with a portion of the cannulas 1010A and 1010B extending into the patient's eye 1001.

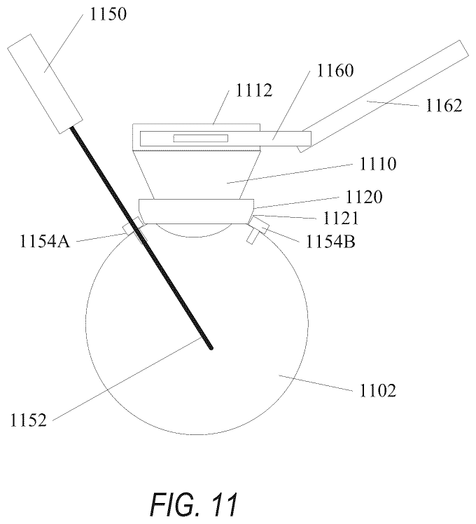

FIG. 11 shows another example of a procedure using ophthalmologic visualization systems as described. An eye 1102 is shown. A viewing lens 1110 similar to viewing lenses described in other examples above is shown. The viewing lens 1110 contacts the eye 1102. An instrument 1150 such as a probe, suction, light source, etc. is shown with a functional end 1152 entering the eye 1102 through a cannula 1154A. The viewing lens 1110 is utilized to visualize a procedure using the functional end 1152 of the instrument 1150.

A contact lens holder 1120 is shown located between the viewing lens 1110 and the eye 1102. The contact lens holder 1120 is shown in FIG. 11 engaging a first cannula 1154A and a second cannula 1154B. Similar to the example of FIG. 5, although two cannulas 1154A, 1154B are shown, three or more cannulas are also within the scope of the invention. Three cannulas provides a stability advantage, as three point will define one and only one circle around a cornea. The use of three cannulas therefore provides a stable fixed location for the contact lens holder 1120. In the example of FIG. 11, a portion of sides of the contact lens holder 1120 are shown with a bevel 1121 that substantially matches an angled side surface of the cannulas 1154A, 1154B. The bevel 1121 aids in stability of the contact lens holder 1120 and helps prevent a possibility of sides of the contact lens holder 1120 undermining one or more of the cannulas 1154A, 1154B and causing them to begin to pull out of the eye 1102.

A fixture 1160 is shown holding the viewing lens 1110. In the example of FIG. 11, the fixture 1160 includes a resilient C-clip that is easily attachable and detachable around an upper portion 1112 of the viewing lens 1110. In one example, the fixture 1160 includes a resilient polymer. In one example, the fixture 1160 includes a spring-deformable metal clip. A handle 1162 is further shown coupled to the fixture 1160. In one method of operation, a surgical assistant uses the handle 1162 to hold the fixture 1160 and the viewing lens 1110 generally in place on the patient's eye 1102. The contact lens holder 1120 also engages the one or more of the cannulas 1154A, 1154B as described above to provide location and support to the viewing lens 1110 in addition to the surgical assistant who is controlling/holding the handle 1162. Because of the additional centering/location assistance provided by the lens holder 1120 engaging the cannulas 1154A, 1154B, the task of the surgical assistant controlling/holding the handle 1162 is much easier. Although the handle 1162 is shown in FIG. 11 coupled to the viewing lens 1110, in other examples, the viewing lens the handle 1162 is alternatively coupled to the contact lens holder 1120.

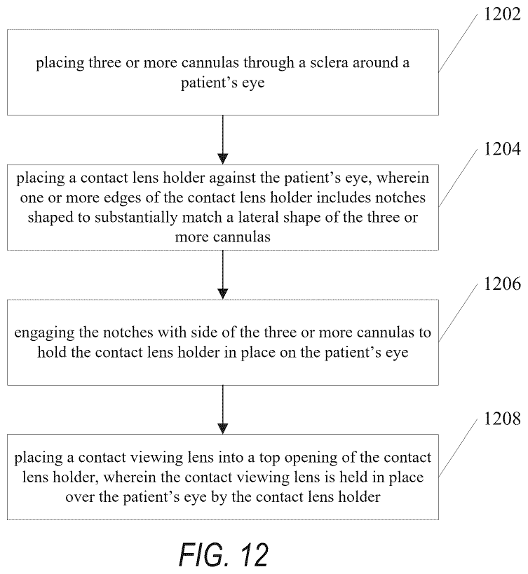

FIG. 12 shows one example of a method of visualizing an interior of a patient's eye. In operation 1202, three or more cannulas are placed through a sclera around a patient's eye. In operation 1204, a contact lens holder is placed against the patient's eye. In operation 1204, one or more edges of the contact lens holder includes notches shaped to substantially match a lateral shape of the three or more cannulas. In operation 1206, the notches are engaged with a side of the three or more cannulas to hold the contact lens holder in place on the patient's eye. In operation 1208, a contact viewing lens is placed into a top opening of the contact lens holder, wherein the contact viewing lens is held in place over the patient's eye by the contact lens holder.

Example configurations and methods as described provide simple, inexpensive components that provide safety and adjustability for a physician or physician assistant during a procedure. Example configurations and methods as described provide an ability to use a contact lens more easily, instead of a non-contact lens. To better illustrate the method and apparatuses disclosed herein, a non-limiting list of aspects is provided here:
Aspect 1. An ophthalmologic visualization system, comprising: a viewing lens adapted to contact a patient's eye; a plurality of cannulas configured for insertion in a limbus of the patient's eye; a contact lens holder, the contact lens holder including; a holder body with a top opening in the holder body sized to fit closely around a bottom portion of the viewing lens; a bottom surface to contact the patient's eye; and a plurality of notches in a side of the holder body, adjacent to the bottom surface, the plurality of notched shaped to substantially match a lateral shape of the plurality of cannulas.
Aspect 2. An ophthalmologic visualization system as disclosed in the present specification.
Aspect 3. A method, comprising: placing three or more cannulas through a sclera around a patient's eye; placing a contact lens holder against the patient's eye, wherein one or more edges of the contact lens holder includes notches shaped to substantially match a lateral shape of the three or more cannulas; engaging the notches with side of the three or more cannulas to hold the contact lens holder in place on the patient's eye; and placing a contact viewing lens into a top opening of the contact lens holder, wherein the contact viewing lens is held in place over the patient's eye by the contact lens holder.

These and other examples and features of the present infusion devices, and related methods will be set forth in part in the above detailed description. This overview is intended to provide non-limiting examples of the present subject matter—it is not intended to provide an exclusive or exhaustive explanation.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. An ophthalmologic visualization system, comprising:
a viewing lens adapted to contact a patient's eye;
a plurality of cannulas configured for insertion in a sclera of the patient's eye;
a contact lens holder, the contact lens holder including;
a holder body with a top opening in the holder body sized to fit closely around a bottom portion of the viewing lens;
a bottom surface to contact the patient's eye; and
a plurality of notches in a side of the holder body, adjacent to the bottom surface, the plurality of notches shaped to substantially match a lateral shape of the plurality of cannulas.

2. The ophthalmologic visualization system of claim 1, wherein the holder body includes an interior shelf configured to contact a bottom of a contact lens.

3. The ophthalmologic visualization system of claim 1, wherein the plurality of notches includes three notches.

4. The ophthalmologic visualization system of claim 1, further including a tubing holder configured to secure a section of tubing that is coupled to the contact lens holder.

5. The ophthalmologic visualization system of claim 1, wherein the tubing holder includes multiple slots.

6. The ophthalmologic visualization system of claim 1, wherein the tubing holder includes an adhesive on a bottom surface to attach the tubing holder to a surgical drape of a patient.

7. The ophthalmologic visualization system of claim 1, wherein sides of holder body include a bevel that matches an angled side surface of the plurality of cannulas when in use in a sclera of the patient's eye.

8. The ophthalmologic visualization system of claim 1, wherein sides of the plurality of notches include a bevel that matches an angled side surface of the plurality of cannulas when in use in a sclera of the patient's eye.

9. The ophthalmologic visualization system of claim 1, wherein the plurality of notches include sets of notches for both a left side eye and a right side eye.

10. The ophthalmologic visualization system of claim 1, wherein the bottom surface of the holder body includes a curved edge.

11. The ophthalmologic visualization system of claim 1, further including a handle.

12. A method, comprising:
placing three or more cannulas through a sclera around a patient's eye;
placing a contact lens holder against the patient's eye, wherein one or more edges of the contact lens holder includes notches shaped to substantially match a lateral shape of the three or more cannulas;
engaging the notches with a side of the three or more cannulas to hold the contact lens holder in place on the patient's eye; and
placing a contact viewing lens into a top opening of the contact lens holder, wherein the contact viewing lens is held in place over the patient's eye by the contact lens holder.
